(19) United States
(12) Patent Application Publication
(10) Pub. No.: US 2005/0277969 A1
(43) Pub. Date: Dec. 15, 2005

(54) Cutting device

(75) Inventors: Harri Happonen, Tampere (FI); Jukka Koljonen, Nokia (FI)

Correspondence Address: BANNER & WITCOFF, LTD., 28 STATE STREET, 28th FLOOR, BOSTON, MA, 02109-9601, US

(73) Assignee: Inion Ltd., Tampere (FI)

(21) Appl. No.: 10/868,339

(22) Filed: Jun. 15, 2004

(51) Int. Cl. A61B 17/32

(52) U.S. Cl. 606/174; 606/79

(57) ABSTRACT

A cutting device for cutting fixation devices used in surgical operations and made of polymer material or polymer composite. The cutting device comprises a blade arrangement, which comprises one or more blades, one or more guides, which comprise a fixation device space, in which the fixation device to be cut is intended to be arranged, means for using the blade arrangement for cutting the fixation device arranged in the guide. The guide is arranged obliquely with respect to the shear plane of blade arrangement so that a cutting surface which is oblique with respect to the longitudinal axis of the fixation device is formed at the tip of the cut fixation device.

### BACKGROUND OF THE INVENTION

The invention relates to a cutting device for cutting an implant screw or pin made of polymer material to a desired length.

Usually fixation devices of different lengths, such as screws and pins, have to be stored in an operating room or in a hospital storage. Screws and pins that dissolve in the body and are used more and more often are supplied to hospitals in sterile packages. These are disposable, which means that once a screw or pin package has been opened, the fixation devices included in it cannot be used in another operation. The disposability further increases the number of fixation devices kept in the storage. It is rather difficult to store exactly the right number of screws and pins of certain lengths so that one does not, on one hand, run out of any kind of fixation device and, on the other hand, the lifetime of the fixation devices does not exceed the use-by-date.

In addition, the large number of fixation devices of different lengths causes problems during the actual operation. After the surgeon has informed the nurse responsible for fixation devices of the suitable length of the fixation device needed, e.g. a screw, the nurse first has to find a sterile bag including screws of the right length, open the bag and install the screw in its installation tool. These stages take easily a minute, even longer.

### BRIEF DESCRIPTION OF THE INVENTION

The object of this invention is to provide a new solution to the problems described above.

According to the present invention, the cutting device comprises a blade arrangement, which comprises one or more blades, one or more guides, which comprise a fixation device space, in which the fixation device to be cut is intended to be arranged, means for using the blade arrangement for cutting the fixation device arranged in the guide, the guide being arranged obliquely with respect to the shear plane of blade arrangement so that a cutting surface, which is oblique with respect to the longitudinal axis of the fixation device, is formed at the tip of the cut fixation device.

An advantage of the invention is that the number of fixation devices that dissolve in the body and are stored in a hospital storage or the like can be reduced radically: only one or two screw lengths/diameter are needed in the storage. This substantially facilitates the storage management and allows to reduce storage costs.

Another advantage is that it is quick to cut the fixation device to the right length in situ. The fixation device can be taken out of its sterile bag and installed in the installation tool even before the surgeon informs the nurse of the required length of the fixation device. After this information, it takes only a few seconds to cut the fixation device to the correct length by the cutting tool. Since dozens of fixation means may be installed during one operation, the time required for the operation can be reduced even by several dozens of minutes.

A further advantage is that the cut tip of the fixation device is asymmetrically oblique with respect to the longitudinal axis of the fixation device. The oblique tip guides the fixation device into a borehole provided for it. Thanks to this guidance phenomenon, the fixation device cut by the cutting device according to the invention can be placed in an open wound even without any guide or tissue protector.

The installation of a screw often requires a tissue protector to protect the tissues surrounding the fixing point of the fixation device against damage. The fixing hole required by the fixation device is drilled and any threads needed are formed in the bone through the tissue protector. The actual fixation device is also installed in the fixing hole through the tissue protector. In this case, the bevelled fixation device tip finds its way to the borehole when the fixation device is screwed. The fixation device can also be installed without the surgeon using the installation tool seeing the installation hole.

A further advantage is that when the fixation device is a screw, the cutting device does not damage the screw threads because during the cutting, the screw is supported adequately in a guide dimensioned optimally with respect to the screw diameter. The basic idea of an embodiment according to the invention is that the cutting device comprises several guides of different lengths to allow cutting of the fixation device to different lengths depending on the guide in which it is installed.

The basic idea of another embodiment of the invention is that the cutting device comprises only one guide with an adjustable length. The guide is provided with a scale for adjusting the guide length according to the need.

### BRIEF DESCRIPTION OF THE FIGURES

The invention will now be described in greater detail in the attached drawings, in which

FIG. 1 is a schematic side view of a cutting device according to the invention,

FIG. 2 is a schematic top view of the cutting device illustrated in FIG. 1,

FIG. 3 is a schematic cross-sectional side view of a detail of the cutting device illustrated in FIG. 1,

FIG. 4 is a schematic perspective view of a second cutting device according to the invention,

FIG. 5 is a schematic top view of the cutting device illustrated in FIG. 4,

FIG. 6 is a schematic cross-sectional side view of the cutting device illustrated in FIG. 4,

FIG. 7 is a schematic perspective view of a third cutting device according to the invention,

FIG. 8 is a schematic end view of the cutting device illustrated in FIG. 7, and

FIG. 9 is a schematic and partly cross-sectional side view of the tip of a screw cut by the cutting device according to the invention in a guide tube.

For the sake of clarity, the figures show the invention in a simplified manner. Like reference numbers identify like elements.

### DETAILED DESCRIPTION OF THE INVENTION

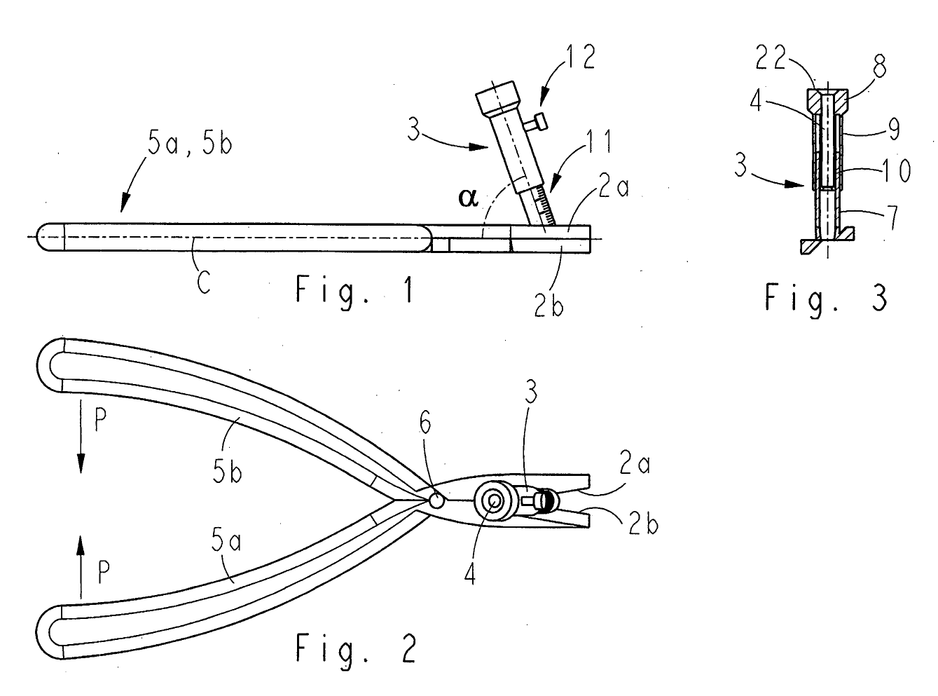

FIG. 1 is a schematic side view of a cutting device according to the invention, FIG. 2 is a top view of the same cutting device, and FIG. 3 is a cross-sectional side view of a detail of the same cutting device. The cutting device 1 resembles side-cutting pliers known per se.

The cutting device 1 includes a blade arrangement, which comprises cutting blades 2a, 2b, which are connected to each other by a joint 6 to allow them to turn with respect to each other. When the blades 2a, 2b are turned with respect to each other about a rotation axis formed by the joint 6, the blades 2a, 2b cut the fixation device to be cut so that the shear plane C is perpendicular to the plane in FIG. 1.

The fixation device is cut by pressing the handles 5a, 5b attached to the blades 2a, 2b against each other in the directions illustrated by arrows P. The cutting device 1 cuts the fixation device obliquely to form angle α between the longitudinal axis L of the fixation device and the cutting surface to be formed at the distal end of the fixation device.

The cutting of the fixation device obliquely is ensured by arranging a guide 3 in the cutting device 1. The guide 2 is arranged in an oblique position, i.e. not perpendicularly, with respect to the shear plane C of the blades 2a, 2b. The cutting angle α is preferably about 60°, but it may advantageously be selected from a range of 30° to 85°.

In the embodiment shown in FIG. 1, the guide 3 comprises an inner guide portion 7 and an outer guide portion 8. The outer guide portion 8 comprises an outer tube 9 and an inner tube 10 arranged coaxially inside it at a distance. The inner guide portion 7 is arranged in a cylindrical space between the outer tube 9 and the inner tube 10, where it can move in the longitudinal direction of the guide 8. Inside the guide 8, there is a space 4 with a round cross-section in which the fixation device to be cut is arranged. The inner guide portion 7 is attached, preferably detachably, to the blade 2a.

The outer guide portion 8 of the guide 3 is slidable with respect to the inner guide portion 7 so that the total length of the guide, i.e. the space 4, can be changed within certain limit values. The length adjustment of the guide 3 may be implemented as stepless or as stepwise. In the former embodiment, the guide 3 length can be adjusted in principle to any length between the limit values, whereas in the latter embodiment, the guide 3 length changes stepwise, e.g. at an interval of 1 mm. The number of such steps between the limit values is at least one, thus allowing selection of the guide 3 length from two alternatives: the maximum length or the minimum length. Usually there are, however, at least two steps between the limit values. Both the stepless adjustment and the stepwise adjustment are known per se and can be implemented in various prior art manners.

The guide 3 is provided with a scale 11, which indicates the length of the fixation device arranged in the guide after it has been cut.

The guide 3 also includes locking means 12 for length adjustment for locking the outer guide portion 8 and the inner guide portion 7 with respect to each other to provide the guide 3 with a desired total length.

By adjusting the length of the guide 3, fixation devices with different lengths and bevelled tips can be prepared, the length of the devices being variable between the minimum and the maximum length defined by the guide portions 7, 8. The minimum length may be 10 mm and the maximum length 40 mm, for instance, but naturally other values are also feasible.

The fixation device to be cut is arranged in the guide 3 so that the distal end of the fixation device, i.e. the end to be bevelled, is pushed into the space 4 for fixation device. The fixation device is pushed into the space 4 provided for it to such an extent that the proximal end of the fixation device, i.e. the end opposite to the distal end, is at a certain point of the guide 3. This may mean, for example, that a fixing screw or pin whose diameter at the proximal end is considerably larger than the diameter of the space 4 for fixation device is pushed into the guide to such an extent that the proximal end meets the upper end 22 of the guide. The fixation device may be attached to the tool, i.e. a screw to a screw driver, at its proximal end for the whole time the fixation device is in the guide 3. On the other hand, a fixation device whose diameter at the proximal end is as large as the diameter of the distal end, such as a headless fixing screw, where the threaded portion reaches from one end of the fixing screw to the other, is arranged in the guide so that the proximal end is on a level with the upper end of the guide 22, for instance. The fixation device can be kept in place in the guide 3 by a tool, for example, which is attached to the counterpart for the tool arranged in the proximal end of the fixation device.

When the fixation device to be cut is arranged in the guide 3 as described above, the fixation device is cut obliquely at the blades 2a, 2b by pressing the handles 5a, 5b in the direction shown by arrows P.

It should be noted here that in another embodiment the guide 3 has a standard length. Cutting device 1 of this kind can be used for cutting fixation devices to a given standard length in a certain and reliable manner. In addition, the cutting device 1 concerned, like any cutting device 1 described in this application, can naturally cut fixation devices to other lengths, too. In that case, the fixation device to be cut is arranged in the guide such that its proximal end is not in contact or on a level with the upper end 22 of the guide but remains at a suitable distance from the upper end 22 of the guide.

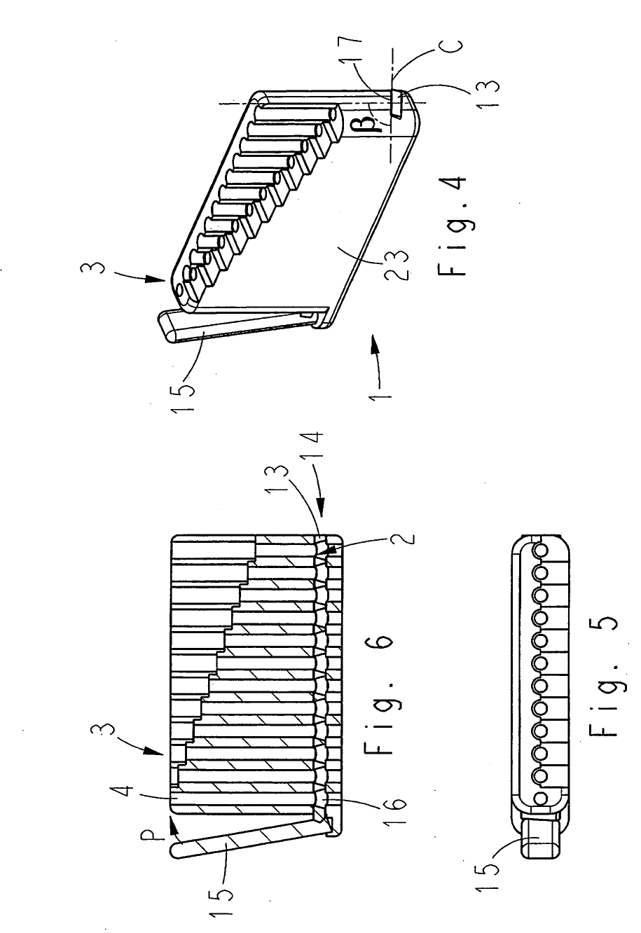

FIG. 4 is a schematic perspective view of a cutting device according to a second embodiment of the invention, FIG. 5 is a top view of the cutting device according to FIG. 4, and FIG. 6 is a cross-sectional side view of the cutting device according to FIG. 4. The cutting device 3 comprises several guides 3 of different lengths arranged one after another in a row in the body 23 of the cutting device.

The cutting device 1 includes a bar 13 with a rectangular cross section arranged to move to and fro with respect to the body 23 to a guide channel 11 provided in the body 23. The bar 13 can be moved with respect to the body 23 by a lever member 15. The lever member 15 is connected to the bar 13 by a joint or the joint between the lever member 15 and the bar 13 is made flexible so that the turning movement of the lever member 15 can be converted into a linear movement of the bar 13.

The bar 13 is arranged at angle β with respect to the longitudinal axis of the guide 3. This angle is preferably 30° to 85°.

The bar 13 is provided with conical through holes 16 at each guide 3. The diameter of the through holes 16 decreases towards the guide 3.

In this embodiment, the blade arrangement of the cutting device 1 is implemented by providing the mouth of each through hole 16 towards the guide 3 with a blade 2, which cuts the fixation device attached to the guide 3 when the bar 13 is moved in the guide channel 14. The blade 2 may be formed by making the edge between the bar 13 surface 17 towards the guide and the through hole 16 sharp.

The lengths and numbers of the guides 3 are preferably such that all lengths of the fixation devices needed in an operation can be formed by the cutting device 1. The guides 3 are provided with a measure or a similar indicator, which indicates the length of the fixation device to be obtained when the fixation device is cut in the guide 3 concerned.

The cutting device shown in FIGS. 4 to 6 comprises 12 guides 3 of different lengths. It is clear that this is only one example of an embodiment according to the invention; the number of guides 3 may naturally be greater or smaller than 12. The cutting device 1 is preferably dimensioned so that it can be used by one hand.

The fixation device to be cut is arranged in a guide 3 with a suitable length as described above. When the lever member 15 is pressed in the direction shown by arrow P, the blade 2 moves with respect to the guide 3 and cuts the fixation device. It should be noted that the movement of the lever member 15 may also be linear.

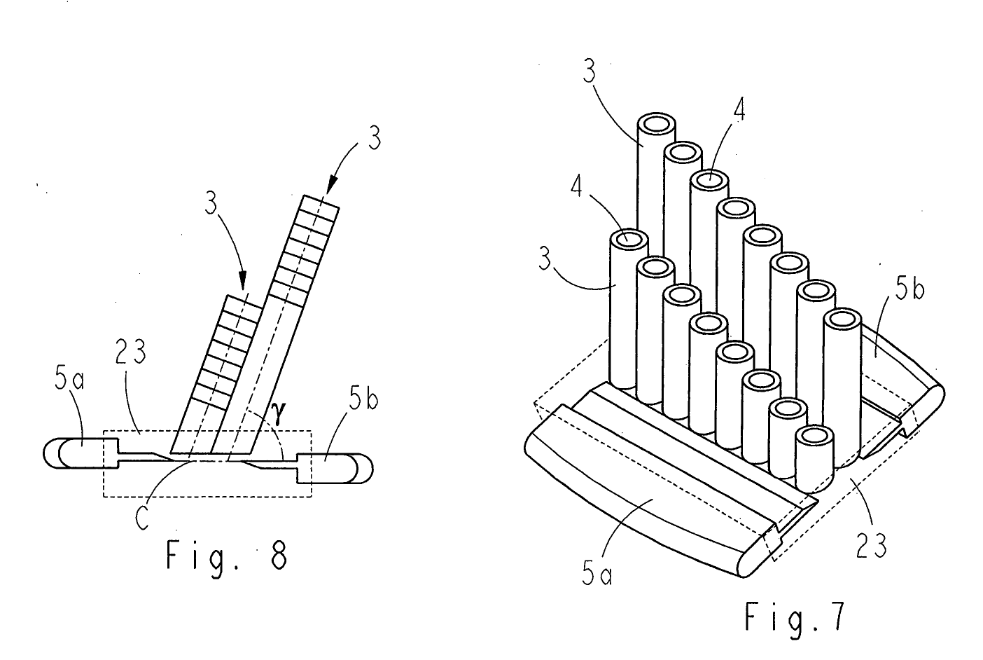

FIG. 7 is a schematic perspective view of a third cutting device according to the invention, and FIG. 8 is a schematic end view of the cutting device illustrated in FIG. 7. The cutting device 1 comprises 16 guides 3 of different lengths, which are arranged in two rows obliquely with respect to the shear plane C of the blades 2a, 2b of the blade arrangement. The angle between the shear plane C and the longitudinal axis of the guides 3 is γ, whose value is preferably between 30° and 85°.

The blades 2a, 2b are straight and arranged movably in the body 23 of the cutting device, to which the guides 3 are also attached.

When the length of the fixation device needed in an operation is known, the fixation device to be cut is arranged in a guide 3 with a suitable length. After this, the fixation device is cut obliquely by pressing the handles 5a, 5b attached to the blades 2a, 2b against each other. The angle that forms at the tip of the fixation device between the cutting surface and the longitudinal axis of the fixation device is γ. Each guide 3 is preferably provided with a measure indicating the length of the fixation device cut in the guide 3.

The cutting device 1 is preferably designed to allow the pressing of the handles 5a, 5b by one hand.

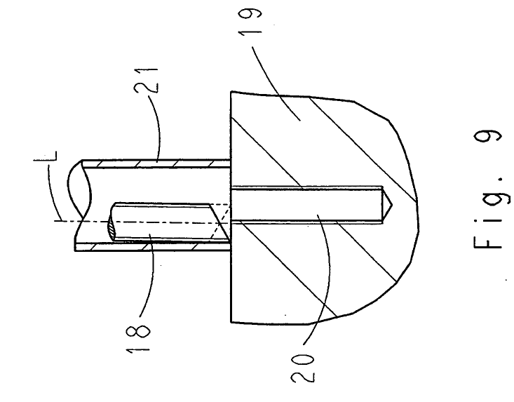

FIG. 9 is a schematic and partly cross-sectional side view of the tip of a screw cut by the cutting device according to the invention in a guide tube. The screw 18 is made of a polymer material suitable for cutting, such as plastic that dissolves in the body, or of a polymer composite containing polymer, such as a plastic-ceramic composite.

The tip of the screw 18, i.e. its distal end, is cut obliquely with respect to its longitudinal axis L. In the case illustrated in FIG. 9, the angle between the cutting surface of the tip and the longitudinal axis L of the screw is approximately 60°.

A fixing hole 20 provided with threads is drilled into tissue 19, which in the example illustrated in FIG. 9 is bony tissue. The drilling and threading are carried out through a guide tube 21 arranged towards the tissue, i.e. a tissue protector. One of the purposes of the guide tube 21 is to protect the surrounding tissue from the tool used in the preparation of the fixing hole 20.

The screw 18 is first attached to the fixing tool, e.g. to a screw driver, by means of which the screw 18 is guided with the aid of the guide tube 21 close to the fixing hole. The diameter of the screw 18 is substantially smaller than the inner diameter of the guide tube 21, for which reason the screw tip is first on the side of the fixing tool 20. When the screw is screwed about its longitudinal axis, its oblique end rotates closer to the centre line of the guide tube 21 and quickly finds its way to the fixing hole 20. It is not necessary for the person installing the screw 18 to see the position of the screw 18 with respect to the fixing hole 20.

The drawings and the related description are only intended to illustrate the inventive concept. The details of the invention may vary within the scope of the claims. Thus the blades of the blade arrangement may be, for example, past-cutting blades, towards-cutting blades or ‘anvil-type blades’. The edges of the past-cutting blades go past each other during cutting. In the towards-cutting blades, the cutting edges of the blades go towards each other during cutting. The anvil-type blade comprises one cutting blade, which during cutting moves towards a substantially blade-free counter surface. The geometry of the blades 2, 2a, 2b may also vary; the blades can be, for example, curved. The blades 2, 2a, 2b may be detachable, in which case worn or damaged blades can be replaced by new ones. The cutting device 1 may comprise a return member which after the cutting of the fixation device, returns the blades 2, 2a, 2b to a position where a new fixation device can be pushed into the guide 3 past the blades 2, 2a, 2b.

The cutting device may also be implemented so that it can be used for cutting fixation devices of different thicknesses. In the embodiment of the cutting device illustrated in FIGS. 1 to 3, for example, the outer guide portion 8 may be detachable and replaceable by a guide portion with a different inner diameter; or the embodiments of the cutting device illustrated in FIGS. 4 to 6 and 7 and 8 may be provided with fixation device spaces 4 with different inner diameters. The thickness of the portion to be cut is typically between 1 and 10 mm in the fixation device.

1. A cutting device for cutting fixation devices used in surgical operations and made of polymer material or polymer composite, the cutting device comprising
a blade arrangement, which comprises one or more blades,
one or more guides, which comprise a fixation device space, in which the fixation device to be cut is intended to be arranged,
means for using the blade arrangement for cutting the fixation device arranged in the guide,
the guide being arranged obliquely with respect to the shear plane of blade arrangement so that a cutting surface, which is oblique with respect to the longitudinal axis of the fixation device, is formed at the tip of the cut fixation device.

2. A cutting device according to claim 1, comprising one guide.

3. A cutting device according to claim 2, wherein the length of the guide is arranged to be adjustable within certain limit values so that fixation devices of different lengths are prepareable by the cutting device.

4. A cutting device according to claim 3, wherein the length of the guide is adjustable steplessly.

5. A cutting device according to claim 3, wherein the guide length is adjustable stepwise.

6. A cutting device according to claim 3, wherein the guide is provided with a scale, which indicates the length of the fixation device to be prepared at the guide.

7. A cutting device according to claim 1, wherein two or more guides with different lengths are arranged obliquely with respect to the shear plane of the blade arrangement.

8. A cutting device according to claim 7, wherein the guides are provided with an identifier, which indicates the length of the fixation devices to be prepared at the guide.

9. A cutting device according to claim 1, wherein the means for using the blade arrangement comprise two handles and the blade arrangement is made to cut the fixation device by changing the mutual position of the handles.

10. A cutting device according to claim 1, wherein the angle between the cutting surface and the longitudinal axis is 30° to 85°.

11. A cutting device according to claim 1, comprising a body and the means for using the blade arrangement of the cutting device comprising a handle, whereby the blade arrangement is made to cut the fixation device by changing the mutual position of the handles.

12. A cutting device according to claim 1, comprising a body and a longitudinal element, which is arranged movably with respect to the body and the guide and which is provided with a through hole, the blade arrangement being formed in connection with the through hole.

13. A cutting device according to claim 1, wherein the blade arrangement comprises a straight blade.

14. A cutting device according to claim 1, wherein the blade arrangement comprises a curved blade.
